(19) United States
(12) Patent Application Publication
(10) Pub. No.: US 2003/0162825 A1
(43) Pub. Date: Aug. 28, 2003

(54) D-amino acid oxidase inhibitors for learning and memory

(75) Inventors: Donald L. Heefner, Hudson, MA (US); Mark G. Currie, Sterling, MA (US); Richard Filip Rossi JR., Norton, MA (US); Charles M. Zepp, Hardwick, MA (US)

Correspondence Address: HESLIN ROTHENBERG FARLEY & MESITI PC, 5 COLUMBIA CIRCLE, ALBANY, NY, 12203, US

(73) Assignee: SEPRACOR INC., Marlborough, MA (US)

(21) Appl. No.: 10/292,368

(22) Filed: Nov. 12, 2002

Related U.S. Application Data
(60) Provisional application No. 60/332,343, filed on Nov. 9, 2001.

(51) Int. Cl. A61K 31/473; A61K 31/404; A61K 31/401; A61K 31/34

(52) U.S. Cl. 514/419; 514/423; 514/461; 514/290

(57) ABSTRACT

Methods and pharmaceutical compositions which inhibit the activity of D-amino acid oxidase (DAO) are disclosed. Inhibition of DAO improves memory, learning and cognition in individuals suffering from neurodegenerative diseases such as Alzheimer's, Huntington's or Parkinson's diseases; the methods and pharmaceutical compositions which inhibit the activity of DAO also improve cognitive dysfunctions associated with aging and improve catatonic schizophrenia. Several genera of heterocycle-2-carboxylic acids are disclosed as DAO inhibitors.

### CROSS REFERENCE TO RELATED APPLICATIONS

This application claims priority from U.S. provisional application, serial No. 60/332,343, filed Nov. 9, 2001, the entire disclosure of which is incorporated herein by reference.

### FIELD OF THE INVENTION

The invention relates to D-amino oxidase inhibitors, and particularly, indole-2-carboxylates, for improving learning, memory and/or cognition.

### BACKGROUND OF THE INVENTION

Memory loss and impaired learning ability are features of a range of clinical conditions. For instance, loss of memory is the most common symptom of dementia states including Alzheimer's disease and senile dementia of the Alzheimer type (the two different terms here distinguish between young and old age onset cases). In fact Alzheimer's disease is the most important clinical entity responsible for progressive dementia in aging populations. In this context, dementia is defined as a syndrome of progressive decline in multiple domains of cognitive function, eventually leading to an inability to maintain normal social and/or occupational performance.

At present, Alzheimer's disease afflicts approximately 4 million Americans. One in ten persons over the age of 65 and nearly half of those over the age of 85 suffer from AD, and AD is the fourth leading cause of death in the U.S. The cost to U.S. society is estimated to be at least $100 billion every year, making AD the third most costly disorder of aging. Alzheimer's disease is manifested as a form of dementia that typically involves progressive mental deterioration, reflected in memory loss, confusion, and disorientation. Pathologically, Alzheimer's Disease can be characterized by thickening, conglutination, and distortion of the intracellular neurofibrils, neurofibrillary tangles and senile plaques composed of granular or filamentous argentophilic masses with an amyloid core. Methods for diagnosing Alzheimer's Disease are known in the art. For example, the National Institute of Neurological and Communicative Disorders and Stroke-Alzheimer's Disease-and the Alzheimer's Disease and Related Disorders Association (NINCDS-ADRDA) criteria can be used to diagnose Alzheimer's Disease (McKhann et al., 1984, Neurology 34:939-944). The patient's cognitive function can be assessed by the Alzheimer's Disease Assessment Scale-cognitive subscale (ADAS-cog; Rosen et al., 1984, Am. J. Psychiatry 141:1356-1364). Alzheimer's disease typically is treated by acetylcholine esterase inhibitors such as tacrine hydrochloride or donepezil.

Early symptoms of AD include memory lapses and mild but progressive deterioration of specific cognitive functions, such as language (aphasia), motor skills (apraxia) and perception (agnosia). The earliest manifestation of AD is often memory impairment, which is required for a diagnosis of dementia in both the NINCDS/ADRDA criteria, which are specific for Alzheimer's disease, and the American Psychiatric Association's Diagnostic and Statistical Manual of Mental Disorders, Fourth Edition (DSM-IV) criteria, which are applicable for all forms of dementia.

Unfortunately, the few forms of treatment for memory loss and impaired learning available at present are not considered effective enough to make any significant difference to a patient, and there is currently a lack of a standard nootropic drug for use in such treatment. There is, therefore, a need for new drugs which are clinically effective in treating memory defects and impaired learning.

Neurodegenerative diseases are diseases in which CNS neurons and/or peripheral neurons undergo a progressive loss of function, usually accompanied by (and perhaps caused by) a physical deterioration of the structure of either the neuron itself or its interface with other neurons. Such conditions include Parkinson's disease, Alzheimer's disease, Huntington's disease and neuropathic pain.

Uncontrollable involuntary movements, psychiatric abnormalities and a loss of intellectual functions (dementia and cognitive decline) are the three major clinical manifestations of Huntington's disease (HD). Involuntary movements, such as chorea, result from abnormalities in the basal ganglia, which regulate motor movements. Dementia and psychiatric abnormalities are due to degeneration of neurons outside the basal ganglia. A loss of neurons in the cerebral cortex (the surface layers of the brain) is particularly prominent in HD. The mechanism of the degeneration is not fully understood. However, the final process of brain cell death appears to be mediated by excitatory amino acids. Cognitive decline, manifested chiefly by loss of recent memory, poor judgement, impaired concentration and acquisition, occur in nearly all patients with HD, but some patients with late-onset chorea never develop dementia. Tasks requiring psychomotor or visuospatial processing, such as skills required by Trail Making B and Stroop Interference Test, are impaired early in the course of the disease and deteriorate at a more rapid rate than memory impairment. The neurobehavioral symptoms typically consist of personality changes, apathy, social withdrawal, agitation, impulsiveness, depression, mania, paranoia, delusions, hostility, hallucinations or psychosis. Depression, commonly seen even in early stages of the disease, is partly biological and partly situational arising from the realization of impending progressive functional impairment; most patients eventually require medical therapy. Tricyclic antidepressants, such as amitriptyline, imipramine and nortriptyline, and serotoninergic agents, such as fluoxetine and sertraline, are used most commonly. The tricyclics, when given at night, have the advantage of helping insomnia and by stimulating appetite they may prevent weight loss, frequently seen in patients with HD. The serotonergic drugs are helpful in patients who, in addition to depression, exhibit obsessive compulsive disorder. Anxiolytics, such as diazepam, alpralozam, and clonazepam, may be helpful to control agitation. Carbamazepine, valproate, and lithium help control manic behavior. Impulse control problems may respond to clonidine or propranolol. Rarely, electroconvulsive therapy is required in patients with medically intractable depression. Psychosis may improve with dopamine receptor blocking drugs (neuroleptics), such as haloperidol, pimozide, fluphenazine and thioridazine, but these drugs can induce tardive dyskinesia and are therefore recommended only if absolutely needed to control symptoms. Clozapine, an atypical antipsychotic drug that does not cause tardive dyskinesia, may be a useful alternative to the typical neuroleptics, but its high cost, risk of agranulocytosis, and other potential side effects limit its use. Neuroleptics are the most effective drugs in the treatment of chorea, but as alluded above, they tend to cause tardive dyskinesia. Monoamine depleting drugs, such as reserpine and tetrabenazine, have the advantage that they do not cause tardive dyskinesia. Tetrabenazine appears to be the most effective suppressant of chorea, but this drug is categorized as investigational and as such is not readily available in the U.S. Both classes of neuroleptics may cause or exacerbate depression, sedation, akathisia and parkinsonism.

Parkinson's disease is a progressive neurological disorder that results from degeneration of dopaminergic neurons, causing the movement impairments that characterize the disease. Often, the first symptom of Parkinson's disease is tremor (trembling or shaking) of a limb, especially when the body is at rest. The tremor often begins on one side of the body, frequently in one hand. Other common symptoms include slow movement (bradykinesia), an inability to move (akinesia), rigid limbs, a shuffling gait, and a stooped posture. Parkinson's disease also causes depression, personality changes, dementia, sleep disturbances, speech impairments, or sexual difficulties. The severity of Parkinson's symptoms tends to worsen over time. Levodopa/carbidopa is probably the single most effective medication for controlling the symptoms of Parkinson's disease. As the loss of dopamine-producing nerve cells continues, symptoms will continue to worsen and the dose of levodopa will often have to be increased. Over time, continual increases in the levodopa dose lead to the development of side effects, some of which may make it impossible to increase the dose any higher. At this point, treatment options become limited. An MAO inhibitor, such as selegiline hydrochloride, or a COMT inhibitor, such as tolcapone or entacapone, may be added to prolong the usefulness of levodopa. Bromocriptine mesylate, pergolide mesylate, amantadine hydrochloride and benztropine mesylate are also used to treat Parkinson's disease. Older ergot dopaminergic agonists were associated with skin inflammation, a tingling sensation in the hands or feet, and lung problems, but newer dopamine agonists, such as pramipexole and ropinirole hydrochloride appear useful alone in the early stages of PD, or together with levodopa to enhance its effect. Quetiapine appears to improve symptoms, and olanzapine improves psychiatric symptoms but worsens motor symptoms.

Schizophrenia, autism, and attention deficit disorder are neuropsychiatric disorders. Clinicians recognize a distinction among neuropsychiatric disorders, and there have been many schemes for categorizing them. The Diagnostic and Statistical Manual of Mental Disorders, Revised, Fourth Ed., (DSM-IV-R), published by the American Psychiatric Association, provides one standard diagnostic system upon which persons of skill rely. It is incorporated herein by reference. According to the framework of the DSM-IV, the mental disorders of Axis I include: disorders diagnosed in childhood [such as Attention Deficit Disorder (ADD) and Attention Deficit—Hyperactivity Disorder (ADHD)] and disorders diagnosed in adulthood. The disorders diagnosed in adulthood include (1) schizophrenia and psychotic disorders; (2) cognitive disorders; (3) mood disorders; (4) anxiety related disorders; (5) eating disorders; (6) substance related disorders; (7) personality disorders; and (8) “disorders not yet included” in the scheme.

ADD and ADHD are disorders that are most prevalent in children and are associated with increased motor activity and a decreased attention span. ADD and ADHD are commonly treated by administration of psychostimulants such as methylphenidate or dextroamphetamine sulfate.

Schizophrenia represents a group of neuropsychiatric disorders characterized by dysfunctions of the thinking process, such as delusions hallucinations, and extensive withdrawal of the patient's interests from other people. Approximately one percent of the worldwide population is afflicted with schizophrenia, and this disorder is accompanied by high morbidity and mortality rates. So called “negative” symptoms of schizophrenia include affect blunting, anergia, alogia and social withdrawal, which can be measured using SANS [Andreasen, 1983, Scales for the Assessment of Negative Symptoms (SANS), Iowa City, Iowa]. “Positive” symptoms of schizophrenia include delusion and hallucination, which can be measured using PANSS (Positive and Negative Syndrome Scale) [Kay et al., 1987, Schizophrenia Bulletin 13:261-276]. “Cognitive” symptoms of schizophrenia include impairment in obtaining, organizing, and using intellectual knowledge which can be measured by the Positive and Negative Syndrome Scale-cognitive subscale (PANSS-cognitive subscale) [Lindenmayer et al., 1994, J. Nerv. Ment. Dis. 182:631-638] or with cognitive tasks such as the Wisconsin Card Sorting Test. Conventional antipsychotic drugs, which act on the dopamine D₂ receptor, can be used to treat the positive symptoms of schizophrenia, such as delusion and hallucination. In general, conventional antipsychotic drugs and atypical antipsychotic drugs, which act on the dopamine D₂ and 5HT₂ serotonin receptor, are limited in their ability to treat cognitive deficits and negative symptoms such as affect blunting (i.e., lack of facial expressions), anergia, and social withdrawal.

“Benign forgetfulness” refers to a mild tendency to be unable to retrieve or recall information that was once registered, learned, and stored in memory (e.g., an inability to remember where one placed one's keys or parked one's car). Benign forgetfulness typically affects individuals after 40 years of age and can be recognized by standard assessment instruments such as the Wechsler Memory Scale (Russell, 1975, J. Consult Clin. Psychol. 43:800-809).

“Close head injury” refers to a clinical condition after head injury or trauma. Such a condition, which is characterized by cognitive and memory impairment, can be diagnosed as “amnestic disorder due to a general medical condition” according to DSM-IV.

Indole derivatives and particularly certain indole-2-carboxylates have been described in the literature for treatment of neurodegenerative disease and neurotoxic injury. EP 396124 A2 discloses indole-2-carboxylates and derivatives for treatment or management of neurotoxic injury resulting from a CNS disorder or traumatic event or in treatment or mamagement of a neurodegenerative disease. Several examples of traumatic events that may result in neurotoxic injury are given, including hypoxia, anoxia, ischemia, associated with perinatal asphyxia, cardiac arrest or stroke. Neurodegeneration is associated with CNS disorders such as convulsions and epilepsy. U.S. Pat. Nos. 5,373,018; 5,374,649; 5,686,461; 5,962,496 and 6,100,289, to Cugola, disclose treatment of neurotoxic injury and neurodegenerative disease using indole derivatives of formula:

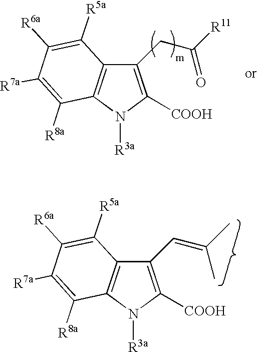

wherein m is 1 to 4; R^{3a} is hydrogen or methyl; R^{5a}, R^{6a}, R^{7a} and R^{8a} are chosen from hydrogen and halogen; and R¹¹ is chosen from hydroxy, lower alkoxy, di(lower alkyl)amino and sulfonamide. None of the above references mention improvement or enhancement of learning, memory or cognition.

### SUMMARY OF THE INVENTION

In one aspect, the invention relates to a method for improving learning and memory comprising administering a D-amino acid oxidase inhibitor. D-amino acid oxidase inhibitors whose activities are demonstrated below include compounds of formula:

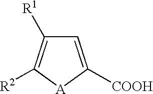

wherein

A is —O— or —NH—;

R¹ is hydrogen or lower alkyl;

R² is hydrogen or lower alkyl; or

taken together R¹ and R² form a six-membered ring, optionally substituted with halogen and/or hydroxyl.

In a second aspect the invention relates to methods for treating a condition chosen from epilepsy, neurotoxic injury, dementia, schizophrenia and neurodegenerative disease comprising administering a therapeutically effective amount of a D-amino acid oxidase (DAO) inhibitor.

### BRIEF DESCRIPTION OF THE DRAWINGS

FIG. 1 is a graph showing acquisition of information in a set of Morris water maze trial where indole-2-carboxylic acid (I-2-C) was administered one hour before the first, second, fourth and sixth training periods.

FIG. 2 is a bar graph showing improved retention of information four days after I-2-C administered as described above for FIG. 1.

FIG. 3 is a graph showing acquisition of information in a set of Morris water maze trial where I-2-C was administered one hour prior to the first training block.

FIG. 4 is a bar graph showing improved retention of information eight days after I-2-C administered as described above for FIG. 3.

FIG. 5 is a graph showing acquisition of information in a set of Morris water maze trial where I-2-C was administered one hour after the last training block.

FIG. 6 is a bar graph showing improved retention of information in the first probe trial after I-2-C administered as described above for FIG. 5.

### DETAILED DESCRIPTION OF THE INVENTION

The invention derives from a discovery that neurodegenerative disorders and deficits in learning and memory can be alleviated by administration of D-amino acid oxidase (DAO) inhibitors. N-methyl-D-aspartate (NMDA)-glutamate receptors are expressed at excitatory synapses throughout the central nervous system (CNS). These receptors mediate a wide range of brain processes, including synaptic plasticity associated with certain forms of memory formation and learning. NMDA-glutamate receptors require binding of two agonists to effect neurotransmission. One of these agonists is the excitatory amino acid L-glutamate, while the second agonist, at the so-called “strychnine-insensitive glycine site”, is now thought to be D-serine. In animals, D-serine is synthesized from L-serine by serine racemase and degraded to its corresponding ketoacid by DAO. Together, serine racemase and DAO are thought to play a crucial role in modulating NMDA neurotransmission by regulating CNS concentrations of D-serine.

The present invention relates to methods and pharmaceutical compositions which inhibit the activity of DAO, thereby improving memory, learning and cognition in individuals suffering from neurodegenerative diseases such as Alzheimer's, Huntington's or Parkinson's diseases; the methods and pharmaceutical compositions which inhibit the activity of DAO also improve cognitive dysfunctions associated with aging and improve catatonic schizophrenia. DAO inhibitors can also be used in conjunction with therapy involving administration of D-serine or an analog thereof, such as a salt of D-serine, an ester of D-serine, alkylated D-serine, or a precursor of D-serine, or can be used in conjunction with therapy involving administration of antipsychotics, antidepressants, psychostimulants, and/or Alzheimer's disease therapeutics. Examples of disorders that can be treated by the methods of the invention include schizophrenia, autism, depression, benign forgetfulness, childhood learning disorders, close head injury, and attention deficit disorder.

In one particular embodiment, the methods of the invention entail administering to a patient a pharmaceutical composition that contains a therapeutically effective amount of a compound of formula I:

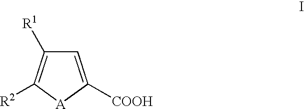

wherein

A is —O— or —NH—;

R¹ is hydrogen or lower alkyl;

R²is hydrogen or lower alkyl; or

taken together R¹ and R² form a six-membered ring, optionally substituted with or more substituents chosen from halogen and hydroxyl.

Patients having a need of therapy for improving or enhancing learning and memory are those exhibiting symptoms of dementia or learning and memory loss. Individuals with an amnesic disorder are impaired in their ability to learn new information or are unable to recall previously learned information or past events. The memory deficit is most apparent on tasks to require spontaneous recall and may also be evident when the examiner provides stimuli for the person to recall at a later time. The memory disturbance must be sufficiently severe to cause marked impairment in social or occupational functioning and must represent a significant decline from a previous level of functioning. The memory deficit may be age-related or the result of disease or other cause. Dementia is characterized by multiple clinically significant deficits in cognition that represent a significant change from a previous level of functioning, including memory impairment involving inability to learn new material or forgetting of previously learned material. Memory can be formally tested by measuring the ability to register, retain, recall and recognize information. A diagnosis of dementia also requires at least one of the following cognitive disturbances: aphasia, apraxia, agnosia or a disturbance in executive functioning. These deficits in language, motor performance, object recognition and abstract thinking, respectively, must be sufficiently severe in conjunction with the memory deficit to cause impairment in occupational or social functioning and must represent a decline from a previously higher level of functioning.

In animals, several established models of learning and memory are available to examine the beneficial cognitive enhancing effects and potential related side effects of treatment. The cognitive enhancing effects are measured by the Morris maze (Stewart and Morris, in Behavioral Neuroscience, R. Saghal, Ed. (IRL Press, 1993) p. 107) the Y-maze (Brits et al., Brain Res. Bull. 6, 71 (1981)), one-way active avoidance test, and two-way passive avoidance test; anxiety-related effects are evaluated in the elevated plus-maze. (Pellow et al., J. Neurosci. Meth. 14:149, 1985.)

The Morris water maze is one of the best-validated models of learning and memory, and it is sensitive to the cognitive enhancing effects of a variety of pharmacological agents (McNamara and Skelton, Brain Res. Rev., 18:33, (1993)). The task performed in the maze is particularly sensitive to manipulations of the hippocampus in the brain, an area of the brain important for spatial learning in animals and memory consolidation in humans. Moreover, improvement in Morris water maze performance is predictive of clinical efficacy of a compound as a cognitive enhancer. For example, treatment with cholinesterase inhibitors or selective muscarinic cholinergic agonists reverse learning deficits in the Morris maze animal model of learning and memory, as well as in clinical populations with dementia (McNamara, 1993). In addition, this animal paradigm accurately models the increasing degree of impairment with advancing age (Levy et al. Pharma. Biochem Behavior 39:781-786 (1991)) and the increased vulnerability of the memory trace to pre-test delay or interference which is characteristic of amnesiac patients.

The test is a simple spatial learning task in which the animal is placed in tepid water, which is opaque due to the addition of powdered milk. The animals learn the location of the platform relative to visual cues located within the maze and the testing room; this learning is referred to as place learning.

As discussed in more detail below, groups of animals receive control solution or a dosage of the therapeutic agent, at the desired time interval prior to training or after training. Control animals typically reach the platform within five to ten seconds after three days of training. The measure of the memory modulator effects of a therapeutic agent is a shift of this time period. Administration of a therapeutic agent results in a dose-dependent increase in availability of synaptic CRF and a behavioral dose-dependent increase in acquisition and memory retention.

The Y-maze test based on visual discrimination is another assay of learning and memory in animals. In this maze, two arms of the maze end in a translucent plastic panel behind which there is a 40-watt electric bulb. The start box is separated from the third arm by a manually-activated guillotine door. In the first trial, all animals are allowed to explore the maze for 5 minutes, and food pellets are available in each arm. On the second day, each animal is placed in the start box with the door closed. When the door is opened, the animal is allowed to move down the arms and eat the pellets which are located in both arms. On the third day, animals receive six trials in groups of three where one arm is closed at the choice point, no discriminative stimulus is present, and two food pellets are available in the open goal box. On days 4-10, a light at the end of the arm with the food pellets is illuminated and ten trials are run, again in groups of three. The time it takes for the animal to reach the food pellets is recorded.

The effectiveness of a therapeutic agent to improve learning and memory in the Y-maze is tested as follows. Fifteen minutes prior to each of the blocks of training trials on days 4-10, groups of animals orally receive control solutions or doses of a ligand inhibitor. Control animals are expected to make 50% correct choices. The measure of efficacy of treatment on memory is an increase in correct responses.

The one-way active avoidance test is another assay of learning and memory in animals. It may be used to assess improvement in age-related memory deficits. An animal is placed in a footshock compartment; an opening door to a safe compartment serves as a signal for avoidance. Briefly, in this test an animal is placed in a Skinner box enclosure that contains a grid floor composed of stainless steel bars. A seven watt light and tone generator at each end of the box serve as conditioned stimuli. A rat or mouse is initially trained by being placed in the footshock compartment facing away from the door. A shock is administered simultaneously with the door opening to the safe compartment. At intervals, the test is repeated, only the shock is delayed for 10 seconds after the door is opened. The time it takes the animal to leave the footshock compartment is recorded.

The effectiveness of a therapeutic agent in improving memory and learning in the one-way avoidance or control solution is tested as follows. Animals are given the therapeutic agent 15 minutes prior to training. Twenty-four hrs later, the groups are tested for retention, without further administration of therapeutic agent. The measure of efficacy is a shortened latency time to leaving the footshock compartment.

The two-way passive avoidance test is another assay of learning and memory. An animal is placed in the safe compartment of the Skinner box and when it enters the footshock compartment, the door is closed and a mild shock is administered. The latency time for entering the second compartment is recorded. Memory is tested 1 to 7 days later. At this time, a shock is not administered.

The effectiveness of a therapeutic agent in improving learning and memory is tested as follows. Immediately prior to training, groups of animals orally receive control solutions or doses of therapeutic agent. Latency time for entering the footshock compartment is then determined.

The elevated plus maze test measures anxiogenic responses in an approach-avoidance situation involving an exposed, lighted space versus a dark, enclosed space. Both spaces are elevated and are set up as two runways intersecting in the form of a plus sign. This type of approach-avoidance situation is a classical test of “emotionality” and is very sensitive to treatments that produce disinhibition and stress. Animals are placed in the center of the maze and are allowed free access to all four arms in a five minute testing period. The time spent in each arm is recorded.

In humans, methods for improving learning and memory may be measured by such tests as the Wechsler Memory Scale and the Minimental test. A standard clinical test for determining if a patient has impaired learning and memory is the Minimental Test for Learning and Memory (Folstein et al., J. Psychiatric Res. 12:185, 1975), especially for those suffering from head trauma, Korsakoff's disease or stroke. The test result serves as an index of short-term, working memory of the kind that deteriorates rapidly in the early stages of dementing or amnesiac disorders. Ten pairs of unrelated words (e.g., army-table) are read to the subject. Subjects are then asked to recall the second word when given the first word of each pair. The measure of memory impairment is a reduced number of paired-associate words recalled relative to a matched control group. Improvement in learning and memory constitutes either (a) a statistically significant difference between the performance of treated patients as compared to members of a placebo group; or (b) a statistically significant change in performance in the direction of normality on measures pertinent to the disease model. Animal models or clinical instances of disease exhibit symptoms which are by definition distinguishable from normal controls. Thus, the measure of effective pharmacotherapy will be a significant, but not necessarily complete, reversal of symptoms. Improvement can be facilitated in both animal and human models of memory pathology by clinically effective “cognitive enhancing” drugs which serve to improve performance of a memory task. For example, cognitive enhancers which function as cholinomimetic replacement therapies in patients suffering from dementia and memory loss of the Alzheimer's type significantly improve short-term working memory in such paradigms as the paired-associate task (Davidson and Stem, 1991). Another potential application for therapeutic interventions against memory impairment is suggested by age-related deficits in performance which are effectively modeled by the longitudinal study of recent memory in aging mice (Forster and Lal, 1992).

The Wechsler Memory Scale is a widely-used pencil-and-paper test of cognitive function and memory capacity. In the normal population, the standardized test yields a mean of 100 and a standard deviation of 15, so that a mild amnesia can be detected with a 10-15 point reduction in the score, a more severe amnesia with a 20-30 point reduction, and so forth (Squire, 1987). During the clinical interview, a battery of tests, including, but not limited to, the Minimental test, the Wechsler memory scale, or paired-associate learning are applied to diagnose symptomatic memory loss. These tests provide general sensitivity to both general cognitive impairment and specific loss of learning/memory capacity (Squire, 1987). Apart from the specific diagnosis of dementia or amnestic disorders, these clinical instruments also identify age-related cognitive decline which reflects an objective diminution in mental function consequent to the aging process that is within normal limits given the person's age (DSM IV, 1994). As noted above, “improvement” in learning and memory within the context of the present invention occurs when there is a statistically significant difference in the direction of normality in the paired-associate test, for example, between the performance of therapeutic agent treated patients as compared to members of the placebo group or between subsequent tests given to the same patient.

The compounds of formula (I), and physiologically acceptable salts and solvates thereof, exhibit an advantageous profile of activity including good bioavailability. These compounds are therefore useful in the treatment or prevention of neurotoxic damage or neurodegenerative diseases. Thus the compounds are useful for the treatment of neurotoxic injury which follows cerebral stroke, thromboembolic stroke, hemorrhagic stroke, cerebral ischemia, cerebral vasospasm, hypoglycemia, amnesia, hypoxia, anoxia, perinatal asphyxia and cardiac arrest. The compounds are also useful in the treatment of chronic neurodegenerative diseases such as: Huntingdon's disease, Alzheimer's senile dementia, amyotrophic lateral sclerosis, multi-infarct dementia, status epilecticus, contusive injuries (e.g. spinal cord injury and head injury), viral infection induced neurodegeneration, (e.g. AIDS, encephalopathies), Down syndrome, epilepsy and schizophrenia.

The invention offers several advantages over many art-known methods for treating neuropsychiatric disorders. For example, unlike many conventional antipsychotic therapeutics, DAO inhibitors can produce a desirable reduction in the cognitive symptoms of schizophrenia. Conventional antipsychotics often lead to tardive dyskinesia (irreversible involuntary movement disorder), extra pyramidal symptoms, and akathesia.

For the purposes of the invention, a D-amino acid oxidase inhibitor is defined as a compound that exhibits an IC₅₀ less than 100 μM against porcine kidney D-amino acid oxidase in the test described herein, in Example 1.

D-amino acid oxidase inhibitors include compounds of formula

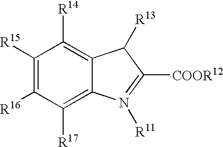

wherein

R¹¹ and R¹² are independently hydrogen, alkyl, substituted alkyl, aryl, or alkylaryl;

R¹³ is hydrogen, alkyl, substituted alkyl, aryl, substituted aryl, alkylaryl or substituted alkylaryl; and

R¹⁴, R¹⁵, R¹⁶ and R¹⁷ are independently hydrogen, hydroxy, halo, amino, cyano, nitro, substituted alkyl, aryl, substituted aryl, arylalkyl, substituted arylakyl, alkoxy;

or pharmaceutically suitable salts or solvates thereof.

Preferred D-amino acid oxidase inhibitors include:

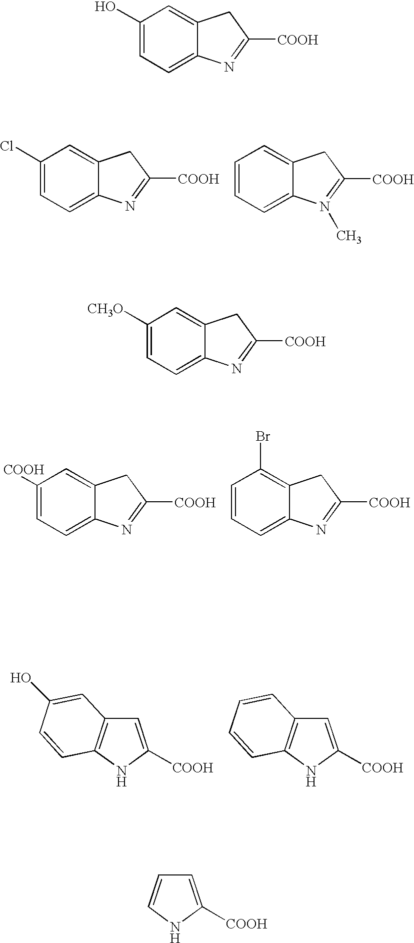

A particularly preferred D-amino acid oxidase inhibitor is 2-indole carboxylic acid.

Other preferred D-amino acid oxidase inhibitors include compounds of formula II:

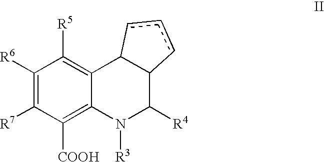

wherein

R³ is hydrogen or methyl;

R⁴ is chosen from alkyl, aryl, substituted alkyl and substituted aryl;

R⁵, R⁶ and R⁷ are chosen independently from hydrogen, halogen, nitro, lower alkyl and lower alkoxy; and

the dashed line bond represents an optional double bond which may be located in either of the two positions shown. Preferred embodiments of this genus have one of the structures depicted in Formulas:

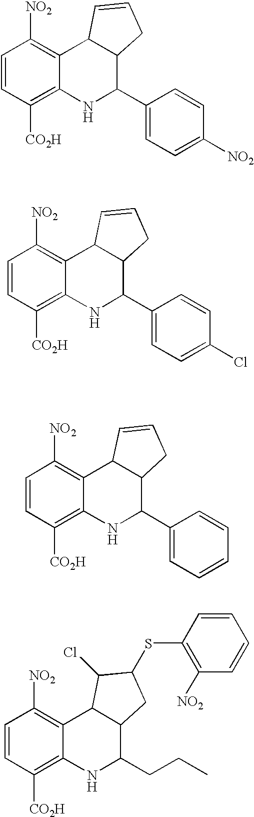

The invention includes compounds above as well as pharmaceutically acceptable salts and solvates of these compounds. The terminology “compound or a pharmaceutically acceptable salt or solvate of a compound” intends the inclusive meaning of “or”, in that a material which is both a salt and a solvate is encompassed.

In a second aspect the invention relates to methods for treating a condition chosen from epilepsy, neurotoxic injury, dementia, schizophrenia and neurodegenerative disease comprising administering a therapeutically effective amount of a D-amino acid oxidase (DAO) inhibitor. Neurodegenerative diseases may include Alzheimer's disease, Parkinson's disease, Huntington's disease, amyotrophic lateral sclerosis, Down syndrome, neuropathic pain, dementia, stroke, mental retardation, ADHD and schizophrenia. Both the first aspect of the invention (learning and memory) and this second aspect envision the use of any and all D-amino acid oxidase (DAO) inhibitors in the method of treatment. However, due to the peculiarities of patent law, and having nothing whatever to do with the scope of the inventors' conception of the invention, certain DAO inhibitors appear from a preliminary search of the literature ineligible to be claimed for the second utility. Thus, for example, indole-2-carboxylic acid, 5-chloroindole-2-carboxylic acid, 5-methoxyindole-2-carboxylic acid and compounds of the generic formula

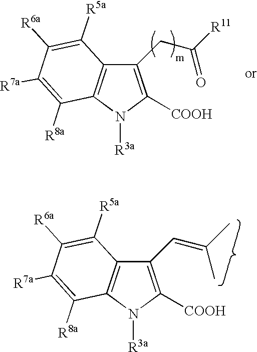

while they are part of the inventive concept, have been excluded from the claims to treating epilepsy, neurotoxic injury, dementia, schizophrenia and neurodegenerative disease. Excluded genera are those wherein m is 1 to 4; R^{3a} is hydrogen or methyl; R^{5a}, R^{6a}, R^{7a} and R^{8a} are chosen from hydrogen and halogen; and R¹¹ is chosen from hydroxy, lower alkoxy, di(lower alkyl)amino and sulfonamide. It may be found upon examination that methods employing certain members of the excluded genera are patentable to the inventors in this application or that additional species and genera not presently excluded are not patentable to the inventors in this application. In either case, the exclusion of species and genera in applicants' claims are to be considered artifacts of patent prosecution and not reflective of the inventors' concept or description of their invention, which encompasses all DAO inhibitors.

In a particular embodiment, DAO inhibitors for treating epilepsy, neurotoxic injury, dementia, schizophrenia, if neurodegenerative disease are compounds of formula

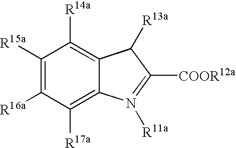

wherein

R^{11a} and R^{12a} are independently hydrogen, alkyl, aryl, or alkylaryl;

R^{13a} is hydrogen, alkyl, substituted alkyl, aryl, substituted aryl, alkylaryl or substituted alkylaryl;

R^{14a}, R^{15a}, R^{16a} and R^{17a} are independently hydrogen, hydroxy, halo, amino, cyano, nitro, carboxy alkyl, substituted alkyl, aryl, substituted aryl, arylalkyl, substituted arylakyl, alkoxy, haloalkyl, or hydroxyalkyl; and

when R^{14a} is carboxy or hydroxy, R^{13a}, R^{15a}, R^{16a} and R^{17a} may not be all hydrogen; when R^{15a} is halogen, methyl or methoxy, R^{13a}, R^{14a}, R^{16a} and R^{17a} may not be all hydrogen; and when R^{16a} is chloro, ^{13a}, R^{14a}, R^{15a}, and R^{16a} may not be all hydrogen.

Preferred compounds include those wherein R^{11a},R^{12a} and R^{13a} are each hydrogen, or wherein R^{14a}, R^{15a}, R^{16a} and R^{17a} are independently hydrogen, hydroxy, halo, alkoxy, or carboxy.

If desired, a pharmaceutical composition containing one or more of the subject DAO inhibitors can be administered to a patient suffering from schizophrenia along with, or in sequence with, a drug for treating schizophrenia (e.g., olanzapine, clozapine, haloperidol, and the like). Similarly, the subject DAO inhibitors can be used in combination with, or in sequence with, other antipsychotics (e.g., “typical,” “atypical,” and depot antipsychotics for treating schizophrenia and other psychotic conditions), psychostimulants (for treating attention deficit disorder, depression, or learning disorders), or Alzheimer's disease therapeutics (for treating Alzheimer's disease). Such pharmaceutical compositions and methods for conjoint therapies are included within the invention.

The phrase “therapeutically effective amount” as used herein means that amount of a compound, material, or composition comprising a compound of the present invention which is effective for producing some desired therapeutic effect by inhibition of DAO in at least a sub-population of cells in an animal and thereby blocking the biological consequences of that pathway in the treated cells, at a reasonable benefit/risk ratio applicable to any medical treatment.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine.

In general, the compounds of the present invention are commercially available or may be prepared by methods well known to persons of skill in the art. In addition methods described below, or modifications thereof, using readily available starting materials, reagents and conventional synthesis procedures may be employed. In these reactions, it is also possible to make use of variants that are in themselves known, but are not mentioned here.

Alkyl is intended to include linear, branched, or cyclic hydrocarbon structures and combinations thereof. Lower alkyl refers to alkyl groups of from 1 to 6 carbon atoms. Examples of lower alkyl groups include methyl, ethyl, propyl, isopropyl, butyl, s-and t-butyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

Alkoxy or alkoxyl refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons.

Aryl means a 5- or 6-membered aromatic ring; a bicyclic 9- or 10-membered aromatic; or a tricyclic 13- or 14-membered aromatic ring system. The aromatic 6- to 14-membered carbocyclic rings include, e.g., benzene, naphthalene, indane, tetralin, and fluorene. Arylalkyl means an alkyl residue attached to an aryl ring. Examples are benzyl, phenethyl and the like.

Substituted alkyl and aryl refer to alkyl or aryl wherein up to three H atoms in each residue are replaced with halogen, haloalkyl, hydroxy, lower alkoxy, carboxy, carboalkoxy (also referred to as alkoxycarbonyl), carboxamido (also referred to as alkylaminocarbonyl), cyano, carbonyl, nitro, amino (primary, secondary or tertiary), alkylamino, dialkylamino, mercapto, alkylthio, sulfoxide, sulfone, acylamino, amidino, phenyl, benzyl, heteroaryl, phenoxy, benzyloxy, or heteroaryloxy.

Many of the compounds described herein may contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, as well as, their racemic and optically pure forms. Optically active (R)- and (S)- isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

While it may be possible for DAO inhibitors to be administered as the raw chemical, it is preferable to present them as a pharmaceutical composition. According to a further aspect, the present invention provides a pharmaceutical composition comprising a compound of formula I or II or a pharmaceutically acceptable salt or solvate thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation. Oral formulations, are well known to those skilled in the art, and general methods for preparing them are found in any standard pharmacy school textbook, for example, Remington: The Science and Practice of Pharmacy. The relevant disclosure is incorporated herein by reference.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein. Oral and parenteral sustained release drug delivery systems are well known to those skilled in the art, and general methods of achieving sustained release of orally or parenterally administered drugs are found, for example, in Remington: The Science and Practice of Pharmacy, chapter 94 of the 19th edition (pages 1660-1675.)

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

Formulations for rectal administration may be presented as a suppository with the usual carriers such as cocoa butter or polyethylene glycol.

Formulations for topical administration in the mouth, for example, buccally or sublingually, include lozenges comprising the active ingredient in a flavored basis such as sucrose and acacia or tragacanth, and pastilles comprising the active ingredient in a basis such as gelatin and glycerin or sucrose and acacia.

Preferred unit dosage formulations are those containing an effective dose, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

### EXAMPLES

### Example 1

### Inhibition Assay/IC₅₀ Determination

Inhibition assays were carried out using Amplex® Red Hydrogen Peroxide Assay Kit (A-22188) obtained from Molecular Probes, Inc., Eugene, Oreg. Porcine kidney D-amino acid oxidase (catalog number A-5222) and D-serine (catalog number S-4250) were obtained from Sigma Company.

A working solution was prepared by mixing: sodium phosphate buffer (8.7 ml, 0.025M, pH 7.4), D-serine solution (1.0 ml, 100 mM in water), horseradish peroxidase (0.2 ml, 200 U/ml in buffer), and Amplex™ Red solution (0.1 ml, 1 mg dye in 200 μl in DMSO (50 μM in DMSO)). A working enzyme solution was prepared by diluting a D-amino oxidase stock solution (65 U/ml) one hundred fold. The working solution (100 μl) was transferred to wells of a Microfluor B microtiter plate and a solution of the inhibitor in DMF was added.

The working enzyme (5 μl) was added to each well and the rate of reaction (hydrogen peroxide released) was determined by measuring the oxidation of Amplex™ Red by spectrophotometry, using a Molecular Dynamics fluorescence plate reader (excitation wavelength 544 nm, emission wavelength, 590 nm) after a reaction time of five minutes. Control experiments were carried out using DMF in the absence of inhibitor. Percent inhibition and IC₅₀ data are shown in Tables 1 and 2 respectively.

TABLE 1

| | | % Inhibition at |
|---|---|---|
| Ex. No. | Structure | 40 μg/mL |
| 1 | 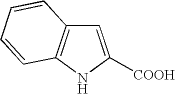 | 93 |
| 2 | 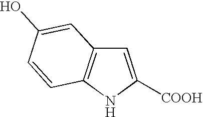 | 90 |
| 3 | 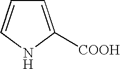 | 87 |
| 4 | 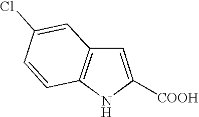 | 86 |
| 5 | 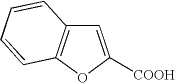 | 85 |
| 6 | 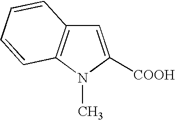 | 58 |
| 7 | 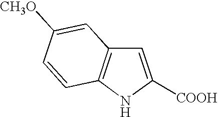 | 32 |

TABLE 2

| Ex. No. | Structure | IC₅₀ (μM) |
|---|---|---|
| 1 | 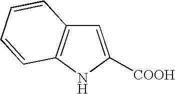 | 2 |
| 8 | 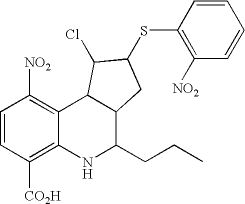 | 11 |
| 9 | 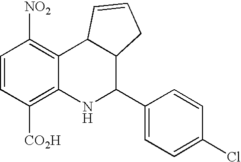 | 15 |
| 10 | 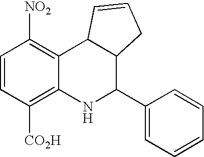 | 17 |
| 11 | 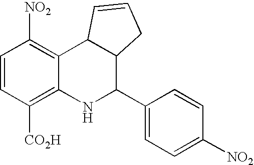 | 29 |

Activity in vivo was examined in the rat swimming model, a standard test which persons of skill recognize as predictive of utility in enhancing learning and memory.

### Example 2

### Enhancement of Learning and Memory by DAO Inhibitors in Animal Model (Morris Water Maze Test)

### Morris Water Maze Task: Protocol

### (Glick, Carlson & Maisonneuve; Oct. 9, 2002)

Training was conducted in a circular pool (160 cm in diameter) filled with water to a depth of 35 cm. The walls of the pool were blue, and the water was made opaque by the addition of approximately 20 ml of white, non-toxic water-based food dye. Water temperature was maintained at approximately 25° C.

During training, rats learn to swim to a submerged platform (approximately 2.5 cm below the surface) constructed of Nalgene with a roughened Plexiglas surface. The surface diameter was approximately 16 cm and its height was approximately 27.5 cm. Experiments were conducted in a darkened room. Lighting of the water-maze was arranged so that illumination of the pool was dim and equivalent in all quadrants. Both acquisition data and retention probe data were collected using Videomex Morris maze software and interface (Columbus Instruments, Columbus, Ohio). The apparatus monitored the animal's progress using a video camera connected to a monitor and a video analysis computer that was outside the view of the swimming animal. Latencies to escape to the platform, the animal's swimming route, and its time spent in the platform-containing quadrant were measured.

Morris maze acquisition training (learning) consisted of 18 trials presented in 6 blocks of 3 trials each. During all training and testing procedures, the experimenter was blind to the animal group membership. To orient the animals to the task, training began with placement of the animal's front paws onto the platform followed by 10 sec of the animal's standing on the platform. Immediately after the third placement, training commenced with the first block of 3 trials. The platform was placed in the center of the southwest quadrant for all training. On each trial, rats were placed into the pool randomly at one of 6 different angular directions. The sequence of placement was counterbalanced between experimental groups. Latency to escape to the platform was measured for each trial. If an animal failed to reach the platform within 180 sec, the animal was placed onto the platform and a latency of 180 sec was recorded. All animals were allowed to remain on the platform for 10 sec after escape or placement. After a block of trials was completed, the animal was returned to an empty tub cage and placed under a heat lamp for 5 min. The animals were given approximately 45 min to dry before being housed individually. Food and water were freely available in the home cage.

Morris maze retention (memory) trials were conducted at 24 hr, 4 days and 8 days after training for each animal. Retention probe trials were conducted by removing the platform and allowing the rat to swim freely in the maze for 60 sec. The amount of time spent in the quadrant previously occupied by the platform and number of crosses over the previous position of the platform were recorded for each animal.

The total sequence of training (Blocks 1-6) and retention testing is shown below.

| | | | | |
|---|---|---|---|---|
| Monday | Tuesday | Wednesday | Thursday | Friday |

1^{st} Week

| | | | | |
|---|---|---|---|---|
| AM: Platform | AM: Block 2 | AM: Block 4 | AM: | AM: Retention |
| Train | | | Block 6 | 24 h |
| PM: Block 1 | PM: Block 3 | PM: Block 5 | | |

2^{nd} Week

| | |
|---|---|
| PM: Retention | PM: Retention |
| day 4 | day 8 |

Drug Administration: Drugs were administered at various times during the protocol. In the first experiment, indole-2-carboxylic acid (I-2-C) (200 mg/kg, ip) was administered four times, one hour before each of the first, second, fourth and sixth training period. In the second, one injection (200 mg/kg) was given one hour prior to the first training block. In the third, one injection (200 mg/kg) was given one hour after the last training block.

Results:

### Example 2.1

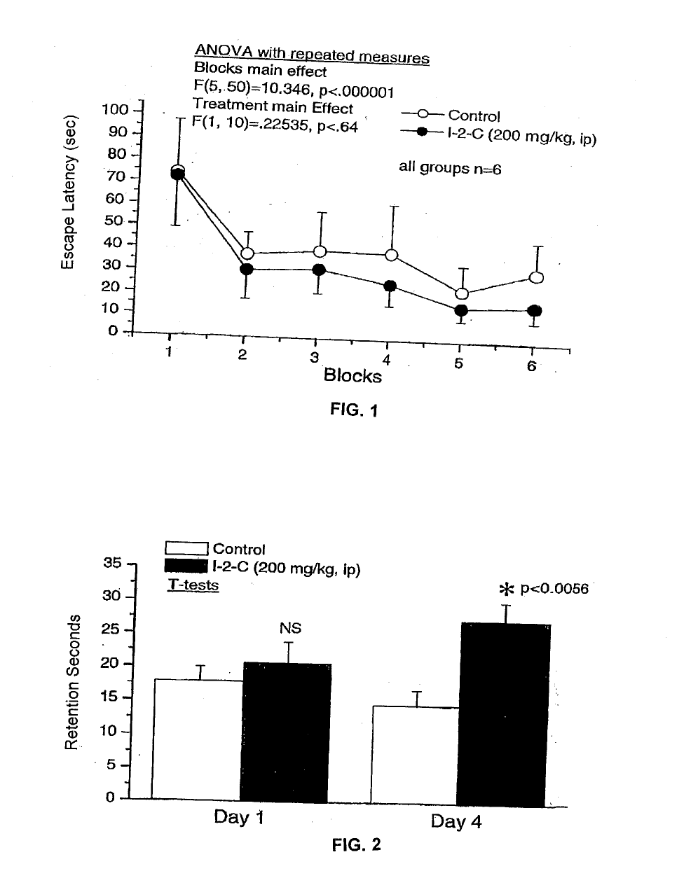

Results of the first set of experiments are shown in FIGS. 1 and 2. FIG. 1 shows escape latency during training. FIG. 2 shows the number of seconds rats kept swimming in the quadrant previously containing the escape platform, a measure of retention. The graphs indicate that treatment with I-2-C improved retention four days after training, and four days after the last injection, and it was concluded that the compound enhanced memory consolidation.

### Example 2.2

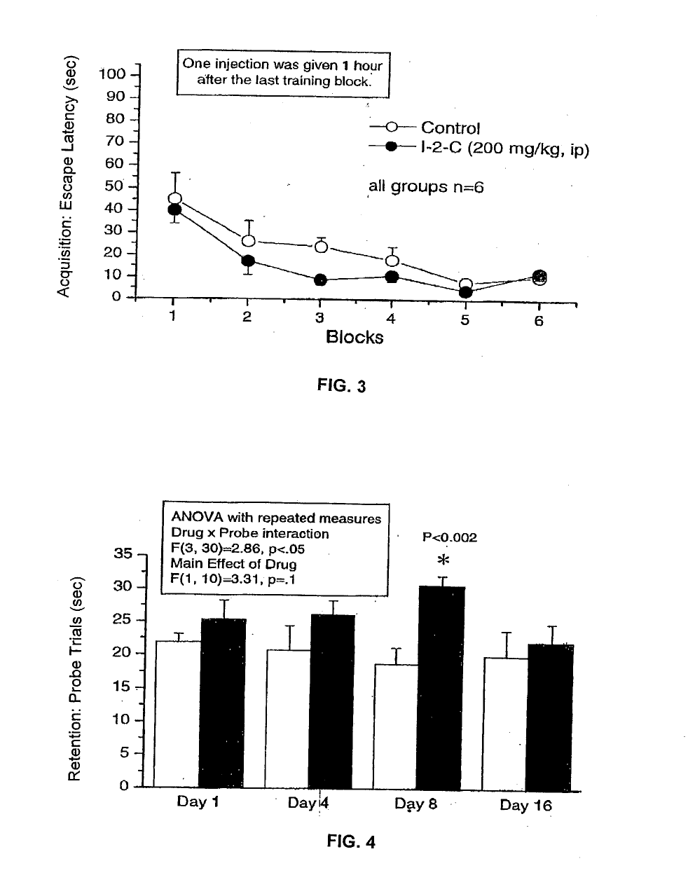

Results of the second set of experiments are shown in FIGS. 3 and 4. FIG. 3 shows escape latency during training, and FIG. 4 shows retention in seconds. The graphs indicate that a single injection of I-2-C administered after training had an improving effect on retention eight days later, but not sixteen days later.

### Example 2.3

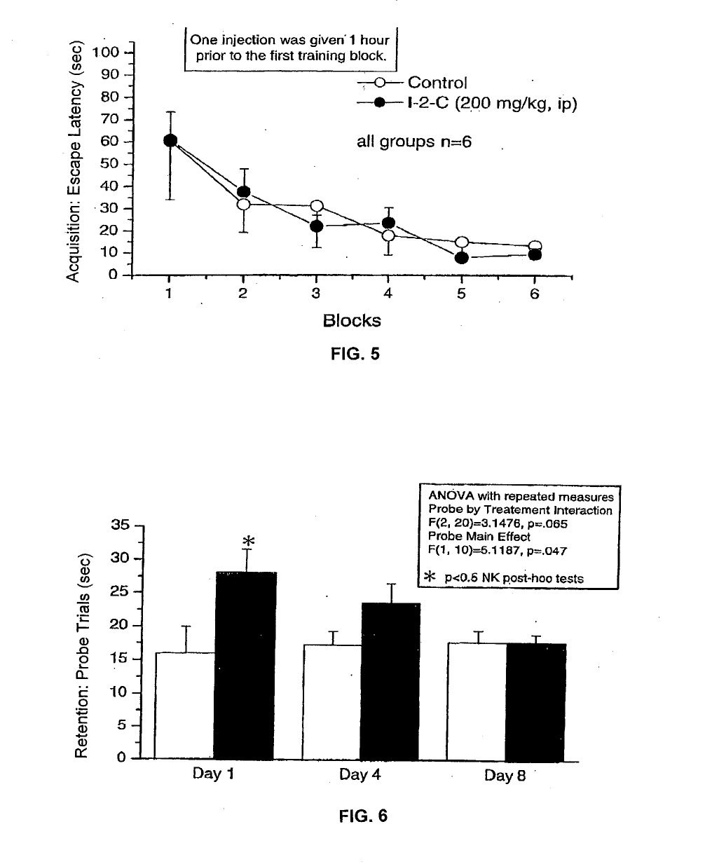

Results of the third set of experiments are shown in FIGS. 5 and 6. FIG. 5 shows escape latency during training, while FIG. 6 shows retention. The graphs indicate that a single injection of I-2-C administered prior to the first training block improved retention during the first probe trial, but not thereafter.

1. A method for improving learning and memory and cognition, or a combination thereof, comprising administering to a mammal an amount of a D-amino acid oxidase inhibitor sufficient to improve learning and memory.

2. A method according to claim 1 wherein said D-amino acid oxidase inhibitor is a compound or a pharmaceutically acceptable salt or solvate of a compound of formula: 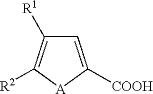
wherein
A is —O— or —NH—;
R¹ is hydrogen or lower alkyl;
R² is hydrogen or lower alkyl; or
taken together R¹ and R² form a six-membered ring, optionally substituted with one or more substituents chosen from halogen and hydroxyl.

3. A method according to claim 1, wherein said D-amino acid oxidase inhibitor is a compound, or a pharmaceutically suitable salt or solvate of a compound of formula: 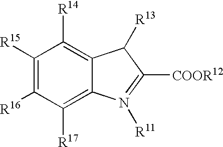
wherein
R¹¹ and R¹² are independently hydrogen, alkyl, substituted alkyl, aryl, or alkylaryl;
R¹³ is hydrogen, alkyl, substituted alkyl, aryl, substituted aryl, alkylaryl or substituted alkylaryl; and
R¹⁴, R¹⁵, R¹⁶ and R¹⁷ are independently hydrogen, hydroxy, halo, amino, cyano, nitro, substituted alkyl, aryl, substituted aryl, arylalkyl, substituted arylakyl, alkoxy.

4. A method according to claim 3 wherein said D-amino acid oxidase inhibitor is a compound or a pharmaceutically acceptable salt or solvate of a compound chosen from:

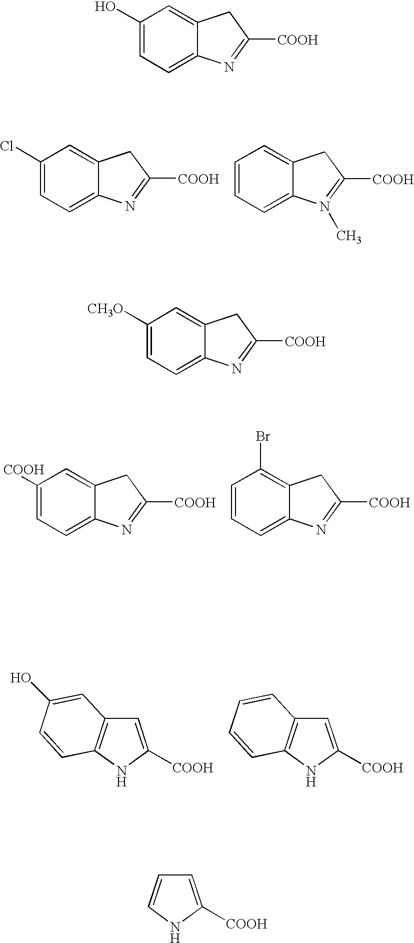

5. A method according to claim 1, wherein the compound administered is indole-2-carboxylic acid.

6. A method according to claim 1 wherein said D-amino acid oxidase inhibitor is a compound or a pharmaceutically acceptable salt or solvate of a compound of formula: 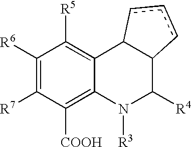
wherein
R³ is hydrogen or methyl;
R⁴ is chosen from alkyl, aryl, substituted alkyl and substituted aryl;
R⁵, R⁶ and R⁷ are chosen independently from hydrogen, halogen, nitro, lower alkyl and lower alkoxy; and
the dashed line bond represents an optional double bond which may be located in either of the two positions shown.

7. A method for treating a condition chosen from epilepsy, neurotoxic injury, dementia, schizophrenia and neurodegenerative disease comprising administering to a patient in need of treatment a therapeutically effective amount of a D-amino acid oxidase (DAO) inhibitor, with the proviso that said DAO inhibitor is not indole-2-carboxylic acid, 5-chloroindole-2-carboxylic acid, 5-methoxyindole-2-carboxylic acid or a compound of the generic formula 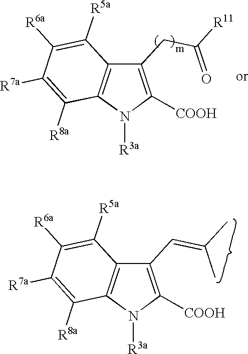
wherein
m is 1 to 4
R^{3a} is hydrogen or methyl;
R^{5a}, R^{6a}, R^{7a} and R^{8a} are chosen from hydrogen and halogen; and
R¹¹ is chosen from hydroxy, lower alkoxy, di(lower alkyl)amino and sulfonamide.

8. A method according to claim 7 wherein said condition is Alzheimer's disease.

9. A method according to claim 7 wherein said condition is schizophrenia.

10. A method according to claim 7, wherein a compound, or a pharmaceutically suitable salt or solvate of a compound of formula: 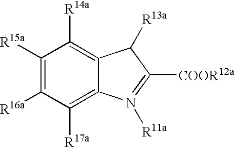
wherein
R^{11a} and R^{12a} are independently hydrogen, alkyl, aryl, or alkylaryl;
R^{13a} is hydrogen, alkyl, substituted alkyl, aryl, substituted aryl, alkylaryl or substituted alkylaryl;
R^{14a}, R^{15a}, R^{16a} and R^{17a} are independently hydrogen, hydroxy, halo, amino, cyano, nitro, carboxy alkyl, substituted alkyl, aryl, substituted aryl, arylalkyl, substituted arylakyl, alkoxy, haloalkyl, or hydroxyalkyl; and
when R^{14a} is carboxy or hydroxy, R^{13a}, R^{15a}, R^{16a} and R^{17a} may not be all hydrogen;
when R^{15a} is halogen, methyl or methoxy, R^{13a}, R^{14a}, R^{16a} and R^{17a} may not be all hydrogen; and when R^{16a} is chloro, R^{13a}, R^{14a}, R^{15a} and R^{16a} may not be all hydrogen.

11. A method according to claim 10, wherein the neurodegenerative condition is selected from Alzheimer's disease, Parkinson's disease, Huntington's disease, amyotrophic lateral sclerosis, Down syndrome, neuropathic pain, dementia, stroke, mental retardation, ADHD and schizophrenia.

12. A method according to claim 10, wherein R^{11a},R^{12a} and R^{13a} are each hydrogen.

13. A method according to claim 10, wherein R^{14a}, R^{15a}, R^{16a} and R^{17a} are independently hydrogen, hydroxy, halo, alkoxy, or carboxy.

14. A method for treating a condition chosen from Parkinson's disease, Alzheimer's disease, Huntington's disease, epilepsy, neuropathic pain, dementia, ADHD and schizophrenia comprising administering to a patient in need of treatment a therapeutically effective amount of a D-amino acid oxidase inhibitor having an IC₅₀ less than 10 μM against porcine kidney D-amino acid oxidase.

15. A method for predicting the utility of a drug candidate for improving learning and memory comprising measuring the activity of said drug candidate in the Morris water maze test.
